(19) United States
(12) Patent Application Publication
(10) Pub. No.: US 2010/0086522 A1
(43) Pub. Date: Apr. 8, 2010

(54) DISPARATE SUICIDE CARRIER CELLS FOR TUMOR TARGETING OF PROMISCUOUS ONCOLYTIC VIRUSES

(75) Inventors: John C. Bell, Ottawa (CA); Anthony T. Power, Nepean (CA)

Correspondence Address: FULBRIGHT & JAWORSKI L.L.P., 600 CONGRESS AVE., SUITE 2400, AUSTIN, TX, 78701, US

(73) Assignee: OTTAWA HEALTH RESEARCH INSTITUTE, Ottawa, ON (CA)

(21) Appl. No.: 12/374,277

(22) PCT Filed: Jul. 18, 2007
(86) PCT No.: PCT/CA07/01268

§ 371 Date: Dec. 10, 2009

Related U.S. Application Data
(60) Provisional application No. 60/807,640, filed on Jul. 18, 2006.

(51) Int. Cl. A61K 35/76; C12N 5/00

(52) U.S. Cl. 424/93.6; 435/346

(57) ABSTRACT

The invention provides compositions and methods for treating neoplastic disease, such as cancer, with an oncolytic virus, such as VSV. A carrier cell is used to target a diseased tissue, and to cloak the oncolytic virus from surveillance by the subject's immune system during a targeting interval. Following delivery of the virus to the target tissue, the lysis of the carrier cell, and of the target cell, by the oncolytic virus, promotes an adaptive tumouricidal immune response. A wide variety of disparate carrier cells may be used, in conjunction with a promiscuous oncolytic virus having broad tropism, in an approach which facilitates successive treatments in which a new carrier will not be susceptible to an adaptive immune response mounted against previously used carriers. The promiscuity of the virus also facilitates lysis of carrier cells and target cells that are allogenic or xenogenic. The lytic phase of the carrier cell infection is staged so that the carrier is administered in an eclipse phase, and lysis follows the conclusion of the therapeutic targeting interval.

### FIELD

The invention is in the field of cancer treatment, particularly oncolytic viral therapies.

### BACKGROUND

A wide variety of oncolytic viruses have been used in preclinical and clinical cancer therapies (see Parato et al., 2005; Bell et al, 2003; Everts and van der Poel, 2005; Ries and Brandts, 2004). For example, an improved therapeutic response has been reported in patients suffering from squamous cell cancer who receive a combination of oncolytic virus therapy and chemotherapy, compared to patients who receive chemotherapy alone (Xia et al., 2004). Oncolytic viruses that have been selected or engineered to productively infect tumour cells include adenovirus (Xia et al., 2004; Wakimoto et al., 2004); reovirus; herpes simplex virus 1 (Shah, et al. 2003); Newcastle disease virus (NDV; Pecora, et al., 2002); vaccinia virus (Mastrangelo et al., 1999; US 2006/0099224); coxsackievirus; measles virus; vesicular stomatitis virus (Stojdl, et al., 2000; Stojdl, et al., 2003); influenza virus; myxoma virus (Myers, R. et al., 2005). For example, EP 1218019, US 2004/208849, US 2004/115170, WO 2001/019380, WO 2002/050304, WO 2002/043647 and US 2004/170607 disclose oncolytic viruses, such as Rhabdovirus, picornavirus, and vesicular stomatitis virus (VSV), in which the virus may exhibit differential susceptibility, particularly for tumor cells having low PKR activity. WO 2005/007824 discloses oncolytic vaccinia viruses and their use for selective destruction of cancer cells, which may exhibit a reduced ability to inhibit the antiviral dsRNA dependent protein kinase (PKR) and increased sensitivity to interferon. WO 2003/008586 similarly discloses methods for engineering oncolytic viruses, which involve alteration or deletion of a viral anti-PKR activity. WO 2002/091997, US 2005/208024 and US 2003/77819 disclose oncolytic virus therapies in which a combination of leukocytes and an oncolytic virus in suspension may be administered to a patient. WO 2005/087931 discloses selected Picornavirus adapted for lytically infecting a cell in the absence of intercellular adhesion molecule-1 (ICAM-1). WO 2005/002607 discloses the use of oncolytic viruses to treat neoplasms having activated PP2A-like or Ras activities, including combinations of more than one type and/or strain of oncolytic viruses, such as reovirus. US 2006/18836 discloses methods for treating p53-negative human tumor cells with the Herefordshire strain of Newcastle disease virus. WO 2005/049845, WO 2001/053506, US 2004/120928, WO 2003/082200, EP 1252323 and US 2004/9604 disclose herpes viruses such as HSV, which may have improved oncolytic and/or gene delivery capabilities.

In many instances, oncolytic viral vectors have been administered by intratumoural injection, such as vectors based on vaccinia virus, adenovirus, reovirus, newcastle disease virus, coxsackievirus and herpes simplex virus (HSV) (Shah et al., 2003; Kaufman, et al. 2005; Chiocca et al., 2004; Harrow et al., 2004; Mastrangelo et al., 1999). Particularly in metastatic disease, a systemic route of delivery for oncolytic viruses may be desirable, for example by intravenous administration (Reid et al., 2002; Lorence et al., 2003; Pecora et al., 2002; Lorence et al., 2005; Reid et al., 2001; McCart et al., 2001).

Although systemic administration of oncolytic viruses may be desirable, this exposes the virus to heightened immune surveillance, which may result in viral inactivation by serum complement components (Ikeda et al., 1999; Wakimoto et al., 2002), uptake by the reticuloendothelial system (Worgall et al, 1997; Ye et al., 2000) or neutralization by circulating antibodies (Ikeda et al., 1999; Hirasawa et al., 2003; Lang et al., 2006; Chen et al., 2000; Tsai et al., 2004). These potential problems are exacerbated by the fact that many potentially useful oncolytic viruses are common human pathogens, or have been in relatively widespread use in human vaccines. It has accordingly been suggested that oncolytic viral therapy might be facilitated by ablation or attenuation of the patient's immune system, which for example occurs during radiation therapy and chemotherapy for cancer (Parato et al., 2005). For example, in mouse tumour model studies with reovirus, HSV and adenovirus oncolytic vectors, it has been shown that antitumour efficacy can be increased by treatment with the chemotherapeutic agent cyclophosphamide, which inhibits neutralizing antibody production (Ikeda et al., 2002; Hirasawa, et al., 2003; Ikeda, K. et al., 1999; Ilan, et al., 1997; Jooss et al., 1996; Kuriyama, et al., 1999; Smith et al., 1996; Wakimoto et al., 2004). US Patent Publication 2006/39894 discloses oncolytic herpes simplex virus strains engineered to counter an innate host immune response. The virus is engineered for expression of the Us11 gene product during the immediate-early phase of the viral life-cycle, preferably without inactivating the Us12 gene, to preserve the ability of the virus to inhibit the host-acquired immune response. Similarly ‘cloaking’ strategies have been proposed to allow a virus to evade the adaptive immune response, such as a vaccinia virus having an extracellular envelope (Ichihashi, 1996) or an adenovirus having a coating of polyethylene glycine or other polymers, or encapsulated with liposomes (Law & Smith, 2001; Fisher, et al., 2001; Holterman et al., 2004; Fukuhara et al., 2003; Eto et al., 2005; Croyle et al., 2001). Viral vectors have also been coaxed to hitchhike on carriers (Cole et al., 2005, Nat Med. 11(10):1073-81).

Various attempts have been made to shield an oncolytic virus from the immune system by delivering the virus in a carrier cell. WO 1999/045783 discloses the use of producer cells harboring an oncolytic virus, including cells that have been rendered incapable of sustained survival, for example by virtue of the cytotoxicity of the oncolytic virus, by exposing the producer cell to radiation or by incorporation of a suicide gene. Similarly, Raykov et al., 2004 disclose the use of inactivated cells, derived from cells having metastatic potential, for targeting oncolytic parvovirus to metastases. The desire to use autologous or syngeneic carrier cells, even in the face of the serious risks that have been recognized to be associated with the use of potentially neoplastic carrier cells, may have arisen out of a perception that this was necessary to avoid an immune response against the carrier cell itself (see also, Garcia-Castro et al., 2005, Tumor cells as cellular vehicles to deliver gene therapies to metastatic tumors, Cancer Gene Therapy (2005) 12, 341-349; and Jevremovic et al., Use of blood outgrowth endothelial cells as virus-producing vectors for gene delivery to tumors, Am. J. Physiol. Heart Circ. Physiol. (2004) 287:H494-H500; U.S. Patent Publication 2005/0208024; and Komarova et al., 2006, Mol Cancer Ther 5(3) 755-766).

### SUMMARY

Various aspects of the invention involve the recognition that in systemic oncolytic viral therapies, the immune system of the patient performs two functions with competing therapeutic results. On the one hand, the oncolytic infection of a tumour may provoke an immune response that is beneficial, in the sense that the immune response may enhance tumoricidal activity. On the other hand, the patient's adaptive immune response to the virus may render successive systemic doses of the virus ineffective, with insufficient virus reaching the tumour to provide a significant oncolytic effect. The invention accordingly recognizes that there are advantages associated with an approach in which delivery of the oncolytic virus avoids the surveillance of the patient's immune system, for the purpose of reliably delivering a targeted oncolytic dosage to a tumour, while enhancing the immune response to the oncolytic infection.

In one aspect, the invention accordingly provides therapeutic methods in which cellular carriers are used to deliver a cloaked oncolytic virus, so that the lysis of the carrier cell releases an infective viral payload in the vicinity of a target cell. In selected embodiments, the disrupted fragments of the carrier cell may then serve as an adjuvant, to stimulate an immune response to the targeted tissue, such as a tumour.

One aspect of the invention relates to the discovery that a very wide variety of carrier cells may be used to effectively deliver an oncolytic virus. In the Examples set out herein, carriers as diverse as human myeloid leukemia cells and insect cells have been used to effectively target an oncolytic virus to cancers in an immuno-competent murine model. The evidence that a broad diversity of carrier cells may be used in this way, including xenogenic carrier cells, indicates that successive oncolytic treatments may be orchestrated with alternative carriers, so that, in some embodiments, on each successive treatment the patient's immune system is naive to the carrier cell. In this way, disparate carrier cell types may be used in sequence so as to escape, in sequence, the adaptive immune response against the previous carrier. For example, a xenogeneic cell line may be used as a carrier, followed by an allogeneic line, or an allogeneic myeloid leukemia carrier followed an allogeneic lymphocytic leukemia cell line. The course of future oncolytic therapy in the patient is accordingly not frustrated by the provocation of an adaptive immune response to the carrier cell, because there is no need to use the same carrier in subsequent treatments. In fact, the provocation of the immune response against the carrier may improve the tumoricidal effect of the oncolytic infection, while helping to ensure that infected carrier cells do not persist in the patient.

In some embodiments, the invention may make use of an oncolytic virus with broad tropism. This broad tropism serves the purpose of facilitating the use of a wide range of carriers, for example in steps of successive administration to a patient where the patient has not yet developed an adaptive immune response to each successive carrier. The use of carriers for an oncolytic virus with broad tropism also serves the purpose of preventing a systemic dose of the virus from being effectively diluted in vivo through widespread adventitious infection in tissues other than the cancerous target. The carrier effectively shields the virus from infectious opportunities, while the carrier delivers the virus to the target.

In various embodiments, the invention may utilize an oncolytic virus that is capable of replicating in a carrier of one species or cell type, and in a tumour or cancer cell of another species or cell type. In this sense, the oncolytic virus of the invention may be said to be ‘promiscuous’, or to exhibit broad tropism. The host range of some oncolytic viruses is broad, such as VSV, while others are more selective. However, recombinant methods of altering viral tropism are known. For example, alterations may be made to a viral coat protein to alter tropism (see for example US Patent Publication 20050221289). Alternatively, a carrier cell may be modified to render it susceptible to viral infection and replication, for example by transformation leading to expression of a membrane ligand or receptor recognized by a particular virus. Alternatively, a method of transduction may be selected that facilitates the infection of a carrier cell by an oncolytic virus that would not normally infect that cell (see for example the following papers and the references cited therein: Liu and Deisseroth, 2006, Blood 107:3027-3033; and, Tan et al., 2007, Mol Med. 13(3-4): 216-226).

In some embodiments, successive treatments of the same patient may be undertaken using alternative carriers. In this way, the adaptive immune response that a host may develop to a particular carrier cell may be avoided in a subsequent round of treatment, using a carrier cell to which the immune system of the patient is naive, i.e. to which the patient does not yet exhibit an adaptive immune response.

### BRIEF DESCRIPTION OF THE DRAWINGS

FIG. 1 illustrates kinetics of a neutralizing antibody (Nab) response.

FIG. 2 is a composite bright-field/fluorescent microscopy image.

FIG. 3 illustrates tumor VSV titers after different treatments in mice.

FIG. 4 shows tumor luciferase activity.

FIG. 5 illustrates a Western blot analysis of VSV protein synthesis.

FIG. 6 is a bioluminescence image showing CT26RLUC distribution.

FIG. 7 shows imaging of therapeutic delivery to lung metastases.

FIG. 8 images VSV delivery using L1210RLUC murine leukemia cells.

FIG. 9 images VSV delivery using human A549 lung carcinoma cells.

FIG. 10 shows infection in lung tumour-bearing hosts.

FIG. 11 shows VSV titre for lung metastases and subcutaneous tumors.

FIG. 12 shows Lung tumor burden in VSV-immune mice.

FIG. 13 images VSV delivery using insect SF-9 cells.

FIG. 14 shows the distribution of systemically administered human leukemia carrier cells in tumor-free or CT26 lung tumor-bearing mice, via in vivo molecular imaging to detect fluc-generated bioluminescence. Localization of Jurkat T-lymphocytic leukemia (A), K562 myeloid leukemia (B), and Meg-01 myeloid leukemia (C) carrier cells are shown.

### DETAILED DESCRIPTION

In various aspects the invention provides methods and compositions for treating a neoplastic disease in a mammalian subject. A carrier cell is used to target an oncolytic virus to a tissue or target cell, so that the carrier cell may be said to be capable of localizing, within a therapeutic targeting interval, to a diseased tissue (the diseased tissue comprising a target cell that is characteristic of the neoplastic disease). The carrier cell may for example be allogenic or xenogenic with respect to the target cell (a factor which carries with it challenges, and benefits).

The carrier cell may be infected with an oncolytic virus to produce an infected carrier cell. The oncolytic virus is selected, or adapted, so that it is capable of productive lytic replication in the infected carrier cell. The characteristics of the carrier cell and the oncolytic virus may be manipulated, so that the lytic replication takes place in the subject following the therapeutic targeting interval, to allow the carrier cell time to target the diseased tissue.

The infected carrier cell may be administered to the subject when the infection of the carrier cell by the oncolytic virus is in an eclipse phase, during which viral antigens are not expressed on surface of the carrier cell. The appropriate selection of the carrier cell, coupled with administration in the eclipse phase, adapts the administration of the carrier cell to the subject so that the subject does not mount an effective adaptive immune response to the infected carrier cell within the therapeutic targeting interval. The carrier cell may be selected or adapted to have an affinity for a neoplastic cell or tissue in the subject, such as a tumour, so that it reaches the target within the targeting interval.

Treatments may be orchestrated so that at least some proportion of the infected carrier cells are lysed by the oncolytic virus in the diseased tissue, following the conclusion of the therapeutic targeting interval. The conclusion of the lytic infection of the carrier cell produces an infective secondary oncolytic virus, which in turn infects and kills target cells by productive lytic replication.

The components and timing of the treatments may be adapted so that lysis of the infected carrier cell is followed by an adaptive immune response to antigenic determinants on the carrier cell. The selection of allogenic or xenogenic carrier cells helps to provoke a meaningful immune response in the subject, which in turn may provide beneficial tumouricidal activity in conjunction with the oncolytic tumouricidal effects mediated by the virus. In addition, the fact that the carrier is allogenic or xenogenic, and hence more easily recognized as non-self by the immune system of the subject, may help to reduce the risk of that a persistent carrier cell population will adversely affect the health of the subject. Accordingly, the invention makes use of a carrier cell to which the subject does not initially have an adaptive immune response (to which the subject is immunologically naive) but to which the host is capable of developing an adaptive immune response.

The ‘cloaking’ of the oncolytic virus by the carrier cell will be particularly advantageous when the subject exhibits an adaptive immune response against antigenic determinants on the oncolytic virus prior to administering the infected carrier cell. For example, if the subject is initially treated with the oncolytic virus, without a carrier cell, and the subject has, as a result, developed an adaptive immune response to the oncolytic virus, then a subsequent treatment with the virus using carrier cells of the invention may be used to help conceal the virus from the immune system during the targeting interval.

In various aspects, the invention may make use of an oncolytic virus with broad tropism, being at the very least able to productively infect both the carrier cell and the cell with which the carrier is allogenic or xenogenic.

The invention may be adapted to facilitate a subsequent step of treatment, utilizing a second carrier cell that is capable of localizing within a second therapeutic targeting interval to the diseased tissue in the subject. The diseased tissue comprising a second target cell that is characteristic of the neoplastic disease. The second carrier cell may be allogenic or xenogenic with respect to the target cell, the second target cell, and with respect to the (first) carrier cell (or wherein the second carrier cell is, in the subject, immunologically distinct from the first carrier cell). In this way, the second carrier cell will not be subject to any adaptive immune response that would be mounted against the first carrier cell. The methods of the invention are used in accordance with the primary treatment, mutatis mutandis. For example, the second oncolytic virus may be the same or different than the (first) oncolytic virus. With the second treatment culminanting in lysis of the second infected carrier cell, followed by an adaptive immune response in the subject against antigenic determinants on the second carrier cell.

In accordance with the foregoing methods, the invention provides for the use of a carrier cell for treating a neoplastic disease, or to formulate a medicament for treating such as disease, in a mammalian subject. The invention also provides corresponding methods for formulating a medicament, involving selecting a carrier cell; infecting the carrier cell in vitro with an oncolytic virus; formulating the medicament for administration to the subject when the infection of the carrier cell by the oncolytic virus is in an eclipse phase; and, adapting the formulation so that lysis of the infected carrier cell by the oncolytic virus in the diseased tissue occurs at the conclusion of the therapeutic targeting interval, to produce an infective secondary oncolytic virus that infects and kills the target cell by productive lytic replication.

Similarly, alternative aspects of the invention provide components for that therapeutic use, such as an infected carrier cell, produced by infecting a carrier cell with an oncolytic virus, for treating a neoplastic disease in a mammalian subject

In one embodiment, the invention provides a method of treating a subject having tumour cells, comprising the step of delivering to the subject a carrier cell containing an oncolytic virus, the carrier cell being immunologically incompatible with the subject, and allogenic or xenogenic with respect to the subject. The subject may be human, and the carrier cell can be an insect cell, a reptile cell, an amphibian cell, an avian cell or a mammalian cell. Exemplary carrier cells include an SF-9 cell, a CT26 cell, an A549 cell, a K562 cell, a Meg-01 cell, a Jurkat cell, or a L1210 cell; and the subject is human. The carrier cell may be an immortalized cell, and/or a tumour cell.

An oncolytic virus may be a replication competent virus, such as a DNA virus, a positive-sense RNA virus, a negative-sense RNA virus, or a double stranded RNA virus. The virus may be released from the carrier cell to infect target cells, such as neoplastic or cancerous cells. The carrier cell may be destroyed by the immune system of the subject, or may be destroyed by the viral replication occurring therein. Optionally, release of the virus occurs prior to or during destruction of the carrier cell. The carrier cell may be delivered to the subject prior to appearance of viral surface antigens on the carrier cell.

According to an embodiment of the invention, the carrier cell is able to pass through small microcapillary bed of the subject. To this end, the carrier cell may be of a small enough size to readily allow can passage through the microcapillary bed. The microcapillary beds in the lungs are an example of a small microcapillary bed through which, optimally, a carrier cell is able to pass.

Further, according to an embodiment of the invention, there is provided a method of preparing an anti-cancer therapeutic composition for administration to a human subject having tumour cells, comprising the steps of: selecting an oncolytic virus; selecting a carrier cell that is immunologically incompatible with a human subject, and is allogenic or xenogenic with respect to the subject; and incubating the virus with the carrier cell for a period of time insufficient to permit presentation of viral surface antigens on the carrier cell. Optionally, the carrier cell may be one that is adapted so that it can pass through a microcapillary bed, such as a pulmonary microcapillary bed in a lung of the subject.

Advantageously, the anti-cancer therapeutic composition disclosed herein, and method of use thereof, can temporarily hide the content of the carrier cell, such as an oncolytic virus, from the complement, reticuloendothelial and immune systems, by employing carrier cells that have been infected with virus or otherwise manipulated ex vivo.

In some embodiments, the carrier cell is a tumour cell that is immunologically incompatible with the subject to be treated and is injected systemically into the subject.

The results presented below show that a major obstacle to oncolytic virus delivery, namely neutralizing antibodies, can be overcome by transiently sequestering virus in infected carrier cells.

In selected embodiments, the oncolytic virus is capable of replicating in the carrier cell and/or capable of replicating in target cells in the subject. Because the carrier cell supports replication of the oncolytic virus, it can produce many copies of the virus. Following administration of the carrier cell to a subject, viruses replicate and lyse the carrier cells, releasing the progeny viruses to infect target cells by, for example, fluid-mediated dispersion or by carrier cell-to-tumour cell contact. Once delivered to target cells in the subject, the oncolytic virus will infect and replicate in the target cells, thereby killing the target cells.

In selected embodiments, the carrier cell is not capable of sustained survival in the body of the subject because it is immunologically incompatible with the subject. In embodiments where the carrier cell is allogenic or xenogenic to the subject, the non-self antigenic determinants on the carrier cell may be recognized by the subject's adaptive immune system, so that the carrier cell is targeted for destruction and cleared from the body.

Oncolytic viruses may be selected based on a natural tropism to tumor cells, or may be manipulated so as to have increased affinity for tumor cells. Viruses may be tailored and/or selected for their ability to replicate preferentially in tumor cells by altering virus tropism with modifications in viral surface antigens to refine cell targeting, by conditionally expressing toxic gene products with tissue-specific gene promoter elements, or by utilizing the ability of a virus to selectively kill tumor cells as a result of cancer-specific defects in the innate antiviral response.

In some embodiments, the invention provides carrier cells derived from a permanent cell line (as opposed to autologous carrier cells derived from a subject). A cultured carried cell can, for example, be engineered to have limited or no innate anti-viral response thus increasing the output of therapeutic virus at the target site. Carrier cells may also be transformed so as to express ligands for tumour antigens on their surface, or ligands that recognize the vasculature of a target tissue (for example to facilitate retention of the carrier cell in tumour beds), or to express antigens, such as tumour antigens, so as to stimulate an immune response to a targeted neoplastic disease, such as a tumour.

As used herein, the term “neoplastic disease” means an abnormal state or condition in a warm-blooded animal characterized by rapidly proliferating cell growth or neoplasm. Neoplastic diseases include malignant or benign neoplasms, including diffuse neoplasms such as leukemia, as well as malignant or benign cancers and tumors (including any carcinoma, sarcoma, or adenoma). A neoplasm is generally recognized as an abnormal tissue that grows by cellular proliferation more rapidly than normal, and can continue to grow after the stimuli that initiated the new growth has ceased. Neoplastic diseases include, for example, tumors such as tumors of the mammary, pituitary, thyroid, or prostate gland; tumors of the brain, liver, meninges, bone, ovary, uterus, cervix, and the like; as well as both monocytic and myelogenous leukemia, adenocarcinoma, adenoma, astrocytoma, bladder tumor, brain tumor, Burkitt lymphoma, breast carcinoma, cervical carcinoma, colon carcinoma, kidney carcinoma, liver carcinoma, lung carcinoma, ovarian carcinoma, pancreatic carcinoma, prostate carcinoma, rectal carcinoma, skin carcinoma, stomach carcinoma, testis carcinoma, thyroid carcinoma, chondrosarcoma, choriocarcinoma, fibroma, fibrosarcoma, glioblastoma, glioma, hepatoma, histiocytoma, leiomyoblastoma, leiomyosarcoma, lymphoma, liposarcoma cell, mammary tumor, medulloblastoma, myeloma, plasmacytoma, neuroblastoma, neuroglioma, osteogenic sarcoma, pancreatic tumor, pituitary tumor, retinoblastoma, rhabdomyosarcoma, sarcoma, testicular tumor, thymoma, Wilms' tumor. Tumors include both primary and metastatic solid tumors, including carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract, (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma) and tumors of the brain, nerves, eyes, and meninges (including astrocytomas, gliomas, glioblastomas, retinoblastomas, neuromas, neuroblastomas, Schwannomas, and meningiomas). In some aspects of the invention, solid tumors may be treated that arise from hematopoietic malignancies such as leukemias (i.e. chloromas, plasmacytomas and the plaques and tumors of mycosis fungoides and cutaneous T-cell lymphoma/leukemia) as well as in the treatment of lymphomas (both Hodgkin's and non-Hodgkin's lymphomas). In addition, treatments of the invention may be useful in the prevention of metastases from the tumors described above.

“Oncolytic virus”, as used herein, refers to any virus, naturally-occurring, engineered or otherwise modified, which is capable of destroying, incapacitating, or reducing the viability of a neoplastic cell, such as a tumor cell. The term “virus” may be used generally to refer either to an oncolytic virus, or to a virus that may not fall within the category of oncolytic viruses.

“Carrier cell”, as used herein, means any cell, naturally-occurring, engineered or otherwise modified, that is used as a delivery vehicle for a virus.

“Autologous”, in its common meaning, means derived or transferred from the same subject; recognized as “self” by the subject's immune system. As used herein, autologous also includes “syngeneic” and refers to a cell that does not elicit a significant immune response when administered to a subject.

“Syngeneic” means genetically identical or closely related, for instance, as to allow tissue transplant; immunologically compatible.

“Allogenic”, or “allogeneic”, as used herein, means genetically different although belonging to or obtained from the same species; allogenic cells are considered to be recognized as “non-self” with respect to a subject's immune system.

“Xenogenic”, or “xenogeneic”, as used herein, means genetically different and belonging to or obtained from a different species; recognized as “non-self” by a subject's immune system.

“Immunologically compatible”, as used herein, means a cell carrying identical or closely related genes; does not elicit a significant immune response in a subject; or well tolerated by a subject's immune system.

“Immunologically incompatible”, as used herein, means a cell carrying different genes; recognized as “non-self” by a subject's immune system; or capable of eliciting an immune response in a subject.

“Immunocompetent”, as used herein, refers to an organism having a complete or at least a partially intact immune system. As used herein, immunocompetent includes immunocompromised and pharmacologically immunosuppressed individuals, and is intended to distinguish from genetically immunodeficient animals, such as SCID mice.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, for example by application following tumour resection, or by application of the composition through a tissue-penetrating non-surgical wound. In particular, parenteral administration is contemplated to include, but is not limited to, subcutaneous, intraperitoneal, intravenous, intraarterial, intramuscular, or intrasternal injection and intravenous, intraarterial, or kidney dialytic infusion techniques.

“Tumour”, as used herein, refers to any type of tumour, including solid tumours or non-solid tumors, dispersed tumors, metastatic or disseminated tumors, or tumor cells from any form of tumour.

The subject to which the treatment according to the invention is directed is an animal having an adaptive immune response, such as a jawed vertebrate. An exemplary subject is a mammal, such as a human.

A target cell is “killed” if it is induced to lyse, if it is induced to undergo apoptosis, or if it is rendered incapable of growing or dividing. A virus is considered cytotoxic with respect to a cell if the virus is able to kill the cell after infecting the cell.

A cell “exhibits binding affinity” for a target cell or tissue if the cell binds to the target cell or tissue with greater affinity than the affinity with which it binds to a non-target cell or tissue, respectively.

An oncolytic virus is “replication-selective” for a particular cell if it is more capable of replicating in that cell than in other cells.

The terms “permanent” and “immortalized” with reference to a cell or cell line are used interchangeably herein to mean a cell or cell line that can be cultured and which replicates in culture for a large number of replications without reaching replicative senescence. A tumor cell or cell line may or may not be immortalized.

As used herein, the term “pharmaceutically acceptable carrier” means a chemical ingredient with which the active ingredient may be combined and which, following the combination, can be used to administer the active ingredient to a subject.

In some embodiments, anti-cancer agents may be used in conjunction with treatments of the invention. Examples of anti-cancer compounds, such as chemotherapeutics, include taxol, vincristine, vinblastine, neomycin, combretastatin, podophyllotoxin, TNF-α, angiostatin, endostatin, vasculostatin, a calcium-flux inducing agent, a calcium ionophore, thrombin, an inflammatory cytokine, or interleukin-4.

An oncolytic virus can be one which is known to exhibit oncolytic activity and which is capable of replicating in a carrier cell without ablating the oncolytic activity of the virus. In an optional embodiment, the oncolytic virus of the invention is able to preferentially replicate in a target or tumour cell of the subject, and is less capable of replicating in a non-target or tumor cell than in the target or tumor cell. For example, the oncolytic virus may be incapable of replicating in a non-target or non-tumor cell.

Suitable oncolytic viruses for use in accordance with the present invention include, but are not limited to, a DNA virus, a positive-sense, negative-sense or double stranded RNA viruses. Reovirus is an exemplary double stranded RNA virus that may be used in accordance with an aspect of the invention. Exemplary negative-sense RNA virus include viruses of the families Orthomyxoviridae, Rhabdoviridae and Paramyxoviridae. Examples of suitable DNA viruses include a Herpesvirus, Adenovirus, Parvovirus, Papovavirus, Iridovirus, Hepadenavirus, Poxvirus, mumps virus, human parainfluenza virus, measles virus or rubella virus. Examples of suitable a positive-sense RNA viruses include a Togavirus, Flavivirus, Picornavirus, or Coronavirus. Examples of suitable negative-sense RNA viruses are Orthomysoviridae, Rhabdoviridae, or Paramyxoviridae including an influenza virus or a vesicular stomatitis virus. Vesicular stomatitis virus is beneficial for use in accordance with the present invention due to its preferential infection of tumour cells. The virus can be a replication competent, replication defective, or non-replication competent. An oncolytic virus may be a naturally-occurring virus or it may be genetically modified, for example, to enhance the tumour or target-homing or tumour or target-killing properties of the virus. Such a virus may also be modified to carry a reporter gene, such as RFP, luciferase (LUC) or green fluorescent protein (GFP).

Vesicular stomatitis virus (VSV) is a virus normally contracted by mammals, mainly livestock, predominantly through sand fly or mosquito bites. It is relatively benign and usually manifests with flu-like symptoms. VSV is the prototypic member of the Vesiculovirus genera of the Rhabdovirus family. The genome of the virus is a single molecule of negative-sense RNA that encodes five major proteins: glycoprotein, matrix protein, nucleoprotein, large protein and phosphoprotein. The G protein mediates both virus attachment to the host cell and fusion of the viral envelope with the endosomal membrane following endocytosis. As a viral therapeutic for cancer treatment, VSV can be injected intravenously such that it accesses a patient's entire body, working its way through the patient's bloodstream preferentially targeting and killing cancer cells, thus making it especially useful for disseminated or metastatic disease.

VSV preferentially infects cancerous cells. One reason for this selectivity is that many cancer cells carry a defect in their interferon signaling pathway, which renders them significantly more sensitive to viral infection than healthy cells. The replication competent VSV replicates in the cancer cells and, once released, goes on to target further cancer cells. Since VSV is highly sensitive to interferon however, healthy cells which mount a successful interferon response are relatively resistant to VSV infection. Thus, VSV and other replication competent viruses are good candidates for use in accordance with the invention.

In selected embodiments, the carrier cell is a tumour cell that is immunologically incompatible with the subject to be treated, and more specifically, is allogenic or xenogenic with respect to the subject for whom the therapeutic composition is intended.

The carrier cell may be modified to incorporate additional factors which prevent prolonged survival of the carrier cell in the subject, or which enhance the tumour or target-homing or tumour or target-killing properties of the therapeutic composition. For example, the carrier cell may further comprise an immunomodulatory molecule, a cytokine, a targeting molecule, a cell growth receptor, an immunoglobulin which is specific for the tumor, a nucleic acid encoding an immunomodulatory molecule, a nucleic acid encoding a cytokine, a nucleic acid encoding a targeting molecule, a nucleic acid encoding a cell growth receptor, a nucleic acid encoding an immunoglobulin which is specific for the tumor, or a combination of any of the above. The cell may either naturally comprise one of these molecules or be engineered to comprise the molecule. The carrier cell may for example be engineered to carry a transgene, for example, a suicide gene, an immunomodulatory gene, and immunostimulatory gene, an antiangiogenic gene, or an oncolytic gene. In some embodiments, carrier cells may express ligands that bind a target or tumor microenvironment molecule. A cell line engineered to express on its surface a ligand for a receptor that is found on the surface of target cells, such as tumor cells, tumor extracellular matrix, or tumor neovasculature, may be employed. Examples of such molecules include, but are not limited to, a membrane-anchored version of a single-chain antibody directed against a tumor cell receptor (such as EGFRvIII, CD38); a membrane-anchored version of RGD-4C peptide, known to target alphav integrins of tumor neovasculature.

In an alternative embodiment of the invention, the carrier cell may comprise thymidine kinase. The carrier cell is incapable of sustained survival in the body of the subject because it is immunologically incompatible and exhibits preferential binding affinity for a tumour cell in a human subject. When the carrier cell is administered to the subject, the carrier cell finds and binds with a tumour cell. When gancyclovir is thereafter administered to the subject, the carrier cell's kinase metabolizes gancyclovir to generate a cytotoxic metabolite which is provided to the tumor cell.

A wide variety of methods are known for targeting moieties, such as therapeutic agents, to tumors or to tumor-associated vasculature (see for example Thorne, Expert Opin Biol Ther. 2007 January; 7 (1):41-51). Many of these methods arise from the growing body of information about tumor-specific ligands. For example, US 2003/185832 discloses methods of targeting agents to tumor-associated endothelial cell markers, and catalogues a wide variety of tumour specific antigenic determinants and corresponding monoclonal antibodies. Ligands for targeting carrier cells of the invention could for example be RGD peptides (including echistatin) that bind tumor neovasculture, antibody molecules (for example binding to VEGF-receptor), or molecules that are expressed on blood cells with inherent tumor-homing activity (as disclosed in the Example herein relating to myeloid cells), or ligands known to be active in tumor-homing macrophages, such as TIE-2. A wide variety of vectors and constructs are available to mediate cell surface expression of tumor-specific ligands. For example, U.S. Pat. No. 6,214,613 discloses expression vectors encoding the variable regions of antibodies, so that the variable regions are expressed in a membrane-bound form on the surface of eukaryotic cells. Accordingly, in some embodiments the carrier cell is not an immune effector cell, but is targeted to the a tumour or to tumour vasculature.

Bi-specific antibodies, and related ligands such as bi-specific single-chain antibodies, may be used to target various agents to a tumour or to tumour vasculature (see for example U.S. Pat. Nos. 7,138,103, 7,052,872, and 7074405). In alternative embodiments of the present invention, a bi-specific antibody, or other bi-specific ligand, may bind to a cell surface antigen on the carrier cell and may also recognize a tumour or tumour vasculature specific antigenic determinant, to target the carrier cell to a tumour or to tumour vasculature.

The carrier cell may be derived from an established permanent cell line, which would for example facilitate use of that carrier cell in different subjects.

The therapeutic composition according to the invention may be administered alone, or in combination or in conjunction with other anti-cancer therapies, such as anti-cancer pharmaceutical agents.

The invention further relates to a method of reducing the viability of or killing tumour or target cells in a subject. This method comprises administering to the subject an immunologically incompatible carrier cell, comprising an oncolytic virus which is capable of replicating in the carrier cell. The carrier cell may be selected so that it is not capable of sustained survival in the body of the subject because it is not immune compatible with the subject. Thus, the carrier cell will be recognized as “non-self” and will be targeted for clearance from the body.

In one embodiment of the invention, the carrier cell is optionally a type of cell known to exhibit binding affinity for selected target cells, such as tumor cells. The carrier cell may for example be a tumour cell, a A549 cell, a K562 cell, a Meg-01 cell, a Jurkat cell, a C126 cell, a L1210 cell, a PA-1 cell, an REN cell, a PER C6 cell, a 293 cell, a melanoma cell, a glioma cell, a teratocarcinoma cell, or a cell of myeloid lineage such as a myeloid leukemia cell. The carrier cell may for example be from a species that is immunologically incompatible with the subject to be treated. For instance, when treating a human subject, an allogenic human cell may be used as a carrier cell, or a xenogenic cell from a different species may be used, such as from mouse, rat, hamster or insect. For example, allogeneic or xenogeneic carrier cells derived from a non-MHC matched human or a non-human cell line can be used. In embodiments in which the carrier cell is allogenic or xenogenic to the subject, the cell will generally not be capable of prolonged survival in the body of the subject, because it will attract an effective immune response in the subject.

In selected embodiments, endothelial progenitor cells (EPCs) may be used as carrier cells. EPCs are readily isolated from peripheral blood mononuclear cells by FACS sorting for high surface expression of either CD34 or Flk1. These cells home to sites of neovascularization in vivo. EPC carriers may for example be obtained from a blood sample, infected with oncolytic virus, and IV injected into the subject for tumor delivery. If necessary, the resistance of normal cells to oncolytic virus infection may be overcome by modifying an EPC through genetic or chemical manipulations.

Peripheral blood mononuclear cell (PBMC) derived carriers can be readily obtained from blood samples and can be infected ex vivo, and delivered to a subject for oncolytic virus delivery. Suitable carriers are those that best support virus replication, produce the highest viral yields and are most efficiently delivered to tumor sites following IV administration. The cell population of interest can be isolated from blood by FACS sorting prior to infection. As they are untransformed, PBMC-derived carriers lack the ability to seed tumor growth upon administration. If necessary, the resistance of normal cells to oncolytic virus infection may be overcome by modifying PBMCs through the genetic or chemical methods.

A carrier cell expressing a tumor-homing chemokine receptor may be used. Such cell lines can be identified by screening primary cells or pre-existing cell lines to compare the expression profiles of specific chemokine or cytokine receptors involved in organ- and/or disease-specific cellular homing. Further, a cell line can be genetically modified to express such a receptor. Cell lines expressing high levels of an endogenous or artificially introduced receptor may deliver oncolytic virus specifically to tumor sites. For example, RANK, the receptor for the soluble chemoattractant RANKL, mediates tumor cell homing to bone (D. H. Jones et al., Nature 2006 Mar. 30; 440(7084):692-6.). Metastasis to bone is a complication seen in many types of cancer. Carrier cells expressing high levels of endogenous or artificially introduced RANK receptor can be used to deliver virus preferentially to sites of metastatic growth. Similarly, the chemokine receptor CXCR4 (via CXCL12 ligand) mediates tumor cell homing to lymph-node and lung, two of the most common sites of cancer metastasis (Muller et al., Nature 2001 Mar. 1; 410(6824):50-6.). In various embodiments, this receptor and/or other receptors involved in leukocyte homing may be expressed on carrier cells.

Auxotrophic cells may be used as carrier cells. A possible concern associated with the systemic administration of transformed carrier cells is that some might persist and give rise to new tumors within the subject. An auxotrophic carrier cell line can circumvent this problem. This is a cell line for which propagation and survival depends on a defined nutrient or chemical not present in the extracellular milieu of a subject's body. Such a line can be readily propagated in vitro when the culture medium is supplemented with a critical ingredient, but upon therapeutic administration the cells are deprived of this ingredient and therefore cannot initiate tumor formation. As an example of an auxotrophic cell, various auxotrophic mutants of the Chinese hamster ovary (CHO) cell line have been isolated. Continued proliferation of each of auxotrophic line requires supplementation with an exogenous nutrient such as proline, putrescine, L-glutamine or mevalonate. Thus an auxotrophic carrier cell line can be maintained in culture in the presence of the appropriate nutrient until it is loaded with oncolytic virus and administered to the subject. Since the required supplement is not available in vivo, any carrier cells escaping virus-mediated oncolysis would be unable to seed new tumors.

Genetic manipulation of carrier cells may be used to enhance oncolytic virus production. The ability of a carrier cell to produce oncolytic virus upon tumor delivery can be enhanced through any number of genetic manipulations. Critical genes mediating innate anti-viral immunity can be targeted for stable knockdown using shRNA vectors. Examples of this include the RNA helicases RIG-I and MDA5 are essential for recognition of RNA virus infection and subsequent of the IFN response (Kato et al., Nature 2006 May 4; 441(7089):101-5). Further, toll-like receptors are required for recognition of various microbial motifs and activate anti-viral defenses, for example, TLR7 recognizes viral ssRNA and activates IFN pathway (Lund et al. PNAS 2004 101: 5598-5603).

Chemical treatment of carrier cells may be used to enhance oncolytic virus production. Carrier cells can be treated with chemical inhibitors of innate anti-viral pathways prior to in vitro infection and therapeutic administration. Examples of this include HDAC inhibitors such as trichostatin A (TSA), valproic acid and sodium butyrate inactivate the interferon anti-viral response (Chang H M et al. PNAS 2004 101: 9578-9583; Nusinzon and Horvath, PNAS 2003 100: 14742-14747) and such inhibitors enhance the sensitivity of cells to infection with oncolytic virus. Treatment with HDAC inhibitors may increase viral yield and therefore increase the effective dose delivered to tumor sites. Example 6, below, illustrates ways in which trichostatin A can be employed.

Tank-binding kinase-1 (TBK-1) is required for activation of the interferon anti-viral response during infection. This protein is a client protein of the molecular chaperone HSP90, and thus geldanamycin, a chemical inhibitor of HSP90, impairs the activation of the IFN system in response to viral infection (Yang et al. Mol. Biol. Cell 2006, 10:1091). Geldanamycin may be used to enhance the viral yield of carrier cells.

Storage of pre-infected carrier cells may be done in any way that is acceptable to those of skill in the art. While each dose of carrier cells may be infected immediately prior to injection into the subject, there are ways in which this could be done in advance that would render oncolytic virus/carrier cell regimens less labor intensive. Pre-infection of a large number of carrier cells at once may be conducted, followed by storage for future use, at which time aliquots could be removed for each subject dose. To this end, pre-infected carrier cells may be frozen at −80 or −120° C. in fetal calf serum supplemented with 10% DMSO. Upon thawing and washing the cells some time later, these cells can be used to deliver oncolytic virus to tumors upon therapeutic administration.

The invention encompasses the preparation and use of medicaments and pharmaceutical compositions comprising the anti-cancer therapeutic composition. Such a pharmaceutical composition may consist of the anti-cancer therapeutic composition alone, in a form suitable for administration to a subject, or may comprise the active ingredient and one or more pharmaceutically acceptable additional ingredients.

The formulations of the pharmaceutical compositions described herein may be prepared by a wide variety of methods, for human or veterinary use. In general, such preparatory methods include the step of bringing the active ingredient into association with a carrier or one or more other accessory ingredients, and then, if necessary or desirable, shaping or packaging the product into a desired single- or multi-dose unit.

An effective amount of an agent of the invention will generally be a therapeutically effective amount. A “therapeutically effective amount” generally refers to an amount effective, at dosages and for periods of time necessary, to achieve the desired therapeutic result, such as lysis of a target cell. A therapeutically effective amount a compound may vary according to factors such as the disease state, age, sex, and weight of the individual, and the ability of the compound to elicit a desired response in the individual. Dosage regimens may be adjusted to provide the optimum therapeutic response. A therapeutically effective amount is also generally one in which any toxic or detrimental effects of the compound are outweighed by the therapeutically beneficial effects.

In alternative embodiments, therapeutic compositions of the invention may be administered to a target cell in vivo, in vitro or ex vivo.

Pharmaceutical compositions according to the invention may be prepared, packaged, or sold in formulations suitable for intravenous, intranasal, intratracheal, intraperitoneal, intratumoral, oral, rectal, vaginal, parenteral, topical, pulmonary, buccal, ophthalmic, or another route of administration. Other contemplated formulations include projected nanoparticles, liposomal preparations, controlled- or sustained-release formulations, liquid and oily suspensions, and immunologically-based formulations. A pharmaceutical composition of the invention may be prepared, packaged, or sold in bulk, as a single unit dose, or as a plurality of single unit doses.

Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin.

A pharmaceutical composition of the invention may also be prepared, packaged, or sold in the form of oil-in-water emulsion or a water-in-oil emulsion. These emulsions may also contain additional ingredients including, for example, sweetening or flavoring agents.

A pharmaceutical composition of the invention may be prepared, packaged, or sold in a formulation suitable for rectal or vaginal administration. Such a composition may be in the form of, for example, a suppository, a retention enema preparation, and a solution for rectal or colonic irrigation.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules, in multi-dose containers containing a preservative, or in single-use devices for auto-injection or injection by a medical practitioner.

Additional ingredients may include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Use of the present invention to treat or prevent a disease condition as disclosed herein, including prevention of further disease progression, may be conducted in subjects diagnosed or otherwise determined to be afflicted or at risk of developing the condition. In some embodiments, for oncolytic therapy, patients may be characterized as having adequate bone marrow function (for example defined as a peripheral absolute granulocyte count of >2,000/mm³ and a platelet count of 100,000/mm³), adequate liver function (for example, bilirubin<1.5 mg/dl) and adequate renal function (for example, creatinine<1.5 mg/dl).

Intratumoral injection, or injection into the tumor vasculature is contemplated for discrete, solid, accessible tumors. Local, regional or systemic administration also may be appropriate. For tumors of >4 cm, the volume to be administered may for example be about 4 to 10 ml, while for tumors of <4 cm, a volume of about 1 to 3 ml may be used. Multiple injections may be delivered as single dose, for example in about 0.1 to about 0.5 ml volumes. Viral particles may be administered in multiple injections to a tumor, for example spaced at approximately 1 cm intervals.

Methods of the present invention may be used preoperatively, for example to render an inoperable tumor subject to resection. Alternatively, the present invention may be used at the time of surgery, and/or thereafter, to treat residual or metastatic disease. For example, a resected tumor bed may be injected or perfused with a formulation comprising an oncolytic virus. The perfusion may for example be continued post-resection, for example, by leaving a catheter implanted at the site of the surgery. Periodic post-surgical treatment may also be useful.

Continuous administration of agents of the invention may be applied, where appropriate, for example, where a tumor is excised and the tumor bed is treated to eliminate residual, microscopic disease. Continuous perfusion may for example take place for a period from about 1 to 2 hours, to about 2 to 6 hours, to about 6 to 12 hours, to about 12 to 24 hours, to about 1 to 2 days, to about 1 to 2 weeks or longer following the initiation of treatment. Generally, the dose of the therapeutic agent via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

Treatments of the invention may include various “unit doses.” A unit dose is defined as containing a predetermined-quantity of the therapeutic composition. A unit dose need not be administered as a single injection but may comprise continuous infusion over a set period of time. Unit dose of the present invention may conveniently be described in terms of plaque forming units (pfu) for a viral construct. Unit doses range from 10³, 10⁴, 10⁵, 10⁶, 10⁷, 10⁸, 10⁹, 10¹⁰, 10¹¹, 10¹², 10¹³ pfu and higher. Alternatively, depending on the kind of virus and the titer attainable, one may deliver 1 to 100, 10 to 50, 100 to 1000, or up to about 10⁴, 10⁵, 10⁶, 10⁷, 10⁸, 10⁹, 10¹⁰, 10¹¹, 10¹², 10¹³, 10¹⁴, or 10¹⁵ or higher infectious viral particles (vp) to the patient or to the patient's cells.

Another aspect of the invention relates to a kit or commercial package comprising compositions of the invention, for example for use in treating a neoplastic disease. Kits and packages of this kind may include instructional material, such as a publication, a recording, a diagram, or any other medium of expression which is used to communicate the intended use of the composition of the invention, for example for treating a neoplastic disease, killing target cells in a subject, for preparing or infecting carrier cells using one or more components of the kit, or for administering the carrier cells or formulations of the invention to a subject. The instructional material may, for example, be affixed to a container which contains a pharmaceutical composition of the invention, or be shipped together with a container which contains the pharmaceutical composition. Alternatively, the instructional material may be shipped separately from the container with the intention that the instructional material and the pharmaceutical composition be used cooperatively by the recipient.

The invention also includes a kit comprising a pharmaceutical composition of the invention and a delivery device for delivering the composition to a subject. By way of example, the delivery device may be a squeezable spray bottle, a metered-dose spray bottle, an aerosol spray device, an atomizer, a dry powder delivery device, a self-propelling solvent/powder dispensing device, a syringe, a needle, a tampon, or a dosage measuring container. The kit may further comprise instructional material as described herein.

Carrier cells may be targeted to tumour cells by any means known in the art, either by being naturally selected for tumor-homing properties or through genetic engineering or other modification. Tumour cells carrying virus will naturally migrate to sites of tumours in vivo, possibly due to chemotactic factors.

A carrier cell may or may not harbour a transgene.

As a platform, a permanent cell line capable of delivering virus offers many advantages over the use of infected autologous cells. Each of the cell lines used in the following examples is an established permanent cell line.

The cell can line can be optimized to possess various desired properties. For example, the cell can be engineered to have limited or no innate anti-viral response thus increasing the output of therapeutic virus at the target site; the cell can be engineered or selected for enhanced viral production, or the cell can be engineered for enhanced homing to tumours. Carrier cells can be made to express tumour antigens on their surface in the ideal context to stimulate immunity or to express surface molecules to facilitate retention in tumour beds.

Distribution of an injected carrier cell throughout the body tissues can be influenced by selecting a carrier cell of a particular physical size. For instance, insect cells, such as Schneider line 2 cells, which are very small, are able to distribute throughout a mouse body without becoming lodged in the microcapillary beds of the lungs as do other larger cell types. Smaller diameter cell lines, such as insect cell lines, may beneficial for therapeutic delivery. In data not presented here, the inventors have shown that L1210 cells have widespread dissemination in animals tested, due at least in part due to their smaller size in comparison to A549.

A number of factors influence the ability of a cell to traverse through capillary beds. Cell size is a factor, considering that typically human capillaries average approximately 8 μm in diameter. Deformability of a cell will have an influence, considering as an analogy, that an 8 μm red blood cells can traverse a 2 μm diameter capillary because it is highly deformable. Further, cell-surface adhesion molecules can influence passage of a cell through a capillary bed, as these molecules can determine retainment of the cell within vascular endothelium. Advantageously, a carrier cell according to the invention will combine small diameter, deformability, and cell adhesion molecules to enable good distribution or passage through capillary beds, and access to tumors disseminated throughout the body.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) and all publications, including but not limited to patents and patent applications, cited in this specification are incorporated herein by reference as if each individual publication were specifically and individually indicated to be incorporated by reference herein and as though fully set forth herein. The invention includes all embodiments and variations substantially as described herein, with reference to the examples and drawings.

### Example 1

### Diverse Cell Types Mediate Trojan Horse Delivery of VSV in Immune-Competent Balb/c Mice

Vesicular Stomatitis Virus (VSV) is an effective oncolytic therapeutic when administered intravenously in a variety of naive mouse cancer models.

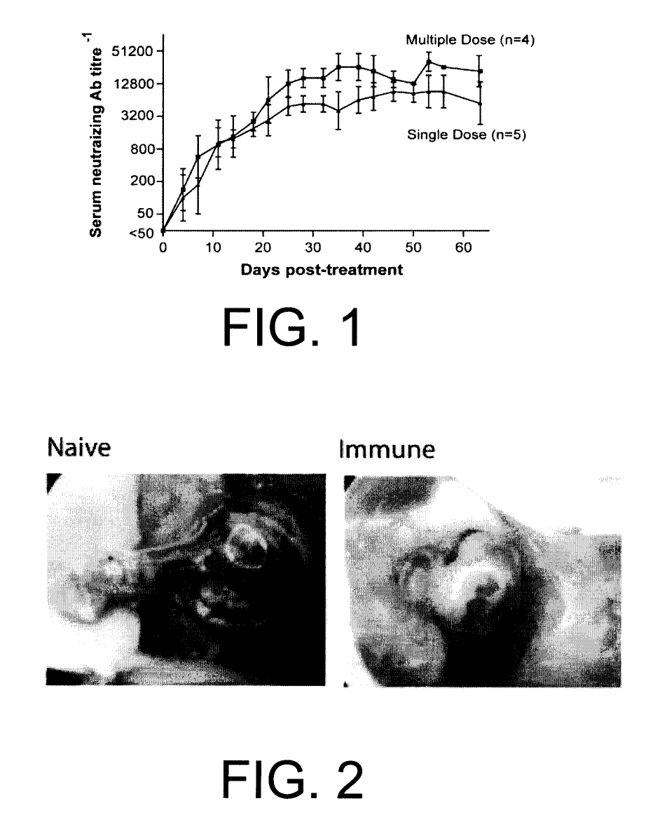

FIG. 1 illustrates serum neutralizing Ab titre up to 60 days post-injection for multiple dose and single dose animals. The kinetics of neutralizing antibody (Nab) response to either single (▴) or multiple (▪) dose therapy with oncolytic VSV are shown. The multiple dose group received 3 intravenous doses of 5×10⁸ pfu per week over a total period of 7 weeks. Geometric mean NAb titres for each group+/−SD are shown.

These data show anti-VSV antibody titers measured from serum samples collected during virus therapy. Neutralizing antibodies were detectable as early as 4 days following the initiation of treatment and continued to rise steadily until reaching plateau levels by 21-28 days. Sub-cutaneous CT-26 tumours were established in mice that had been pre-immunized with VSV and once palpable tumours were detectable, 5×10⁸ pfu of VSV-GFP was administered intravenously.

FIG. 2 illustrates composite bright-field/fluorescent microscopy images of subcutaneous tumors from either naïve (left) or pre-immunized (right) mice, 24 h following intravenous administration of 5×10⁸ VSV-GFP. These data demonstrate that, in naïve mice, extensive virus directed GFP expression was detected in tumors 24 hours after infusion and was correlated with high levels of virus replication within the tumour. On the other hand, virus delivered systemically was unable to reach tumour sites and replicate in mice with circulating NAbs.

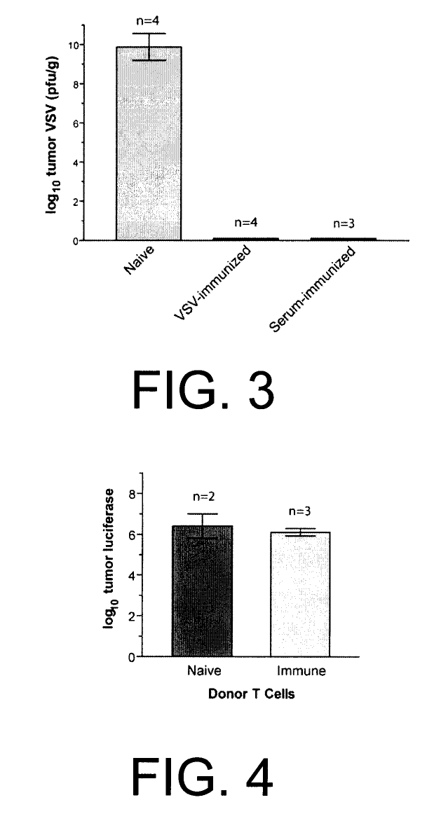

FIG. 3 shows a quantitative analysis of VSV titers in subcutaneous tumors resected from either naïve (left-most bar), VSV-immunized (middle bar), or serum-immunized (right-most bar) mice 24 h following intravenous therapy as described above. Bars represent mean log₁₀ titers+/−SD.

FIG. 4 shows a quantitation of tumor luciferase activity in mice receiving a transfer of T lymphocytes from either naïve (dark bar) or VSV-immune (light bar) donors 24 h prior to intravenous therapy with VSV-luciferase. Tumors were resected and assayed 24 h post-treatment. Bars represent mean relative luciferase units+/−SD.

Passively transferred antibodies but not immune T cells were responsible for the inhibition of VSV delivery to tumour sites consistent with earlier observations that in the mouse, immunity to VSV is largely a humoral response.

Transiently hiding VSV in an infected cellular vehicle or “Trojan Horse”, to enable systemic delivery in the face of neutralizing antibodies was evaluated. To be able to visualize both an intravenously administered cellular vehicle and the viral payload that it delivers, cell lines were created that express renilla luciferase from an integrated cellular gene (CT26^{RLUC}, A549^{RLUC} or L1210^{RLUC}) and a viral strain (VSV^{FLUC}) that expresses firefly luciferase from a viral promoter. For these and subsequent experiments, lung or subcutaneous tumours were established in Balb/c mice, using murine colon cancer cells that were not tagged with luciferase.

### Example 2

### Trojan Horse Delivery of an Oncolytic VSV to Lung Tumors

CT26 colon carcinoma cells were delivered to mice to investigate distribution of cells to animals.

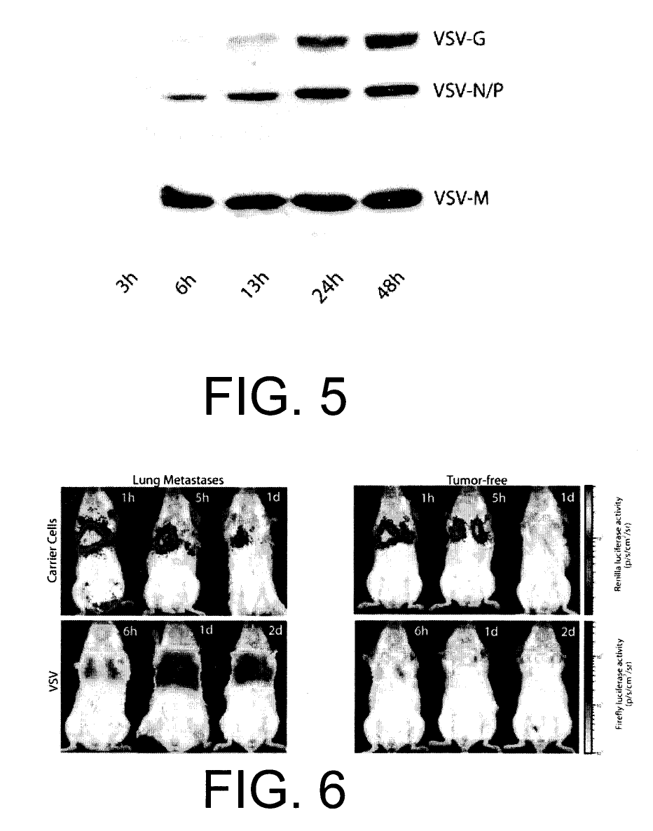

FIG. 5 illustrates a Western blot analysis showing timecourse of VSV protein synthesis in CT26 colon carcinoma cells in vitro. Early on in the infection (as shown at hour 6), there is little synthesis of VSV, but this synthesis increases and extensive synthesis is illustrated between the 13 h and 48 h time points.

FIG. 6 shows bioluminescence imaging, revealing distribution of CT26^{RLUC} carrier cells following in vitro infection with VSV^{FLUC} and subsequent systemic infusion (10⁶ cells/mouse). At indicated time points (from 1 h to 2 d), carrier cell- and virus-driven luciferase activity were imaged following administration of the appropriate coelenterazine (upper panels) or luciferin (lower panels) substrate. Mice were wither bearing metastatic CT26 lung tumors (left panels), or were tumor free (right panels).

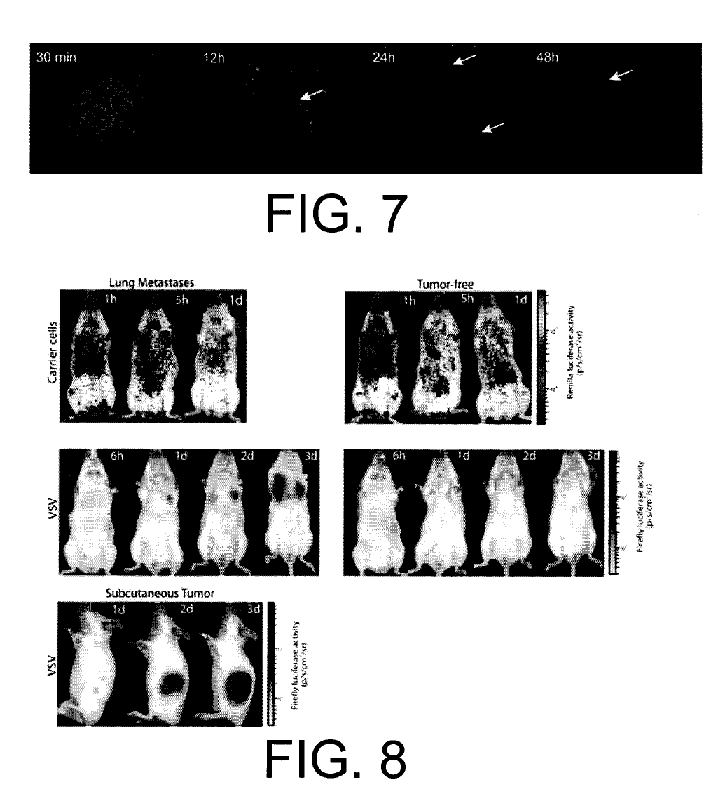

FIG. 7 illustrates two-color fluorescent imaging of trojan horse delivery to lung metastases. The cellular fluorochrome CFSE was used to label CT26 trojan horse cells prior to infection with VSV-RFP and subsequent systemic infusion as above. At indicated timepoints, mice were sacrificed and lungs were examined under a fluorescent dissection microscope. Images shown are composites from CFSE (green, cellular label) and RFP (red, virus replication) channels. Lungs were examined under bright field to identify tumor nodules (arrows).

These data show that, early in the infection of the Trojan Horse carrier cells in vitro (CT26 cells), there are no detectable VSV proteins. However, by 6 hours there is extensive viral protein synthesis and production of infectious virus particles. CT26^{RLUC} cells were therefore infected in vitro for 3 hours and then, before viral antigens would appear on the cell surface, infected CT26^{RLUC} cells were injected intravenously in either tumour free or tumour bearing animals. Shortly after the infusion of the infected carrier cells, either the substrate for renilla luciferase (to detect cells, false coloured blue green) or the firefly substrate (to detect virus replication, false coloured yellow red) was used to detect the expression of the corresponding enzyme using IVIS imaging.

In the first 60 minutes, the majority of the renilla signal (emanating from the cellular gene) is found in the area of the lungs in both tumour bearing and tumour free mice. This is not surprising as the extensive capillary bed of the lung likely acts as a physical restriction to the passage of the CT26^{RLUC} cells. In both sets of animals (tumour free and tumour bearing) the renilla signal begins to abate by five hours (eight hours post infection) and is largely gone by 24 hours as the cells succumb to virus infection. In the tumour free animals, the virus-encoded firefly luciferase signal parallels the cellular renilla signal and is eventually extinguished as the carrier cells are destroyed by the VSV infection, or alternatively are destroyed by the host immune system when the cells are recognized as xenogenic.

In contrast, in the tumour bearing animals the VSV encoded firefly signal continues to increase in intensity in the tumour laden lungs long after the cellular signal is lost (2 days no renilla signal detected data not shown). This signal is largely if not entirely restricted to tumour nodules within the lung.

### Example 3

### Trojan Horse Delivery of VSV Harbouring RFP Gene

Trojan horse carrier cells were labeled with the fluorochrome CFSE and then infected with VSV harbouring the RFP gene (cancer cell). Microscopic examination of lungs following treatment revealed that cells can be found diffusely within the lungs (green signal) but the virus replication (red signal) is eventually restricted to tumour nodules. Immune-Competent Balb/c mice were used.

Two other cell lines were examined for the potential to deliver virus to tumour laden organs. Human A549^{RLUC} lung carcinoma cells behaved very similar to the CT26^{RLUC} cells in that they appeared to be trapped early post-infusion in lung capillaries and were able to effectively deliver virus to tumour beds.

FIG. 8 illustrates data from a murine leukemia cell line, L1210^{RLUC}. This cell line was tested since both the colon cancer and lung cancer cell lines seemed to be retained in the lung, thus the murine leukemia cell line, L1210^{RLUC} was tested. As shown in FIG. 8, this cell line was more diffusely distributed in treated animals, suggesting that perhaps physical trapping of this cell was less important to its targeting to particular organs. The virus encoded signal was also initially diffuse as it emanated primarily from the L1210^{RLUC} carrier cells but with time, the VSV^{FLUC} signal became restricted to tumours in the lung or subcutaneous sites and lost entirely from mice lacking tumours. Dual-enzyme bioluminescent imaging of VSV delivery to lung or subcutaneous CT26 tumors and in tumor-free mice are depicted. Following systemic infusion of 10^{6 L}1210^{RLUC} murine leukemia cells infected with VSV^{FLUC}, carrier cell and virus biodistributions were imaged by administering either coelenterazine (upper panels) or luciferin (lower panels) substrate, respectively.

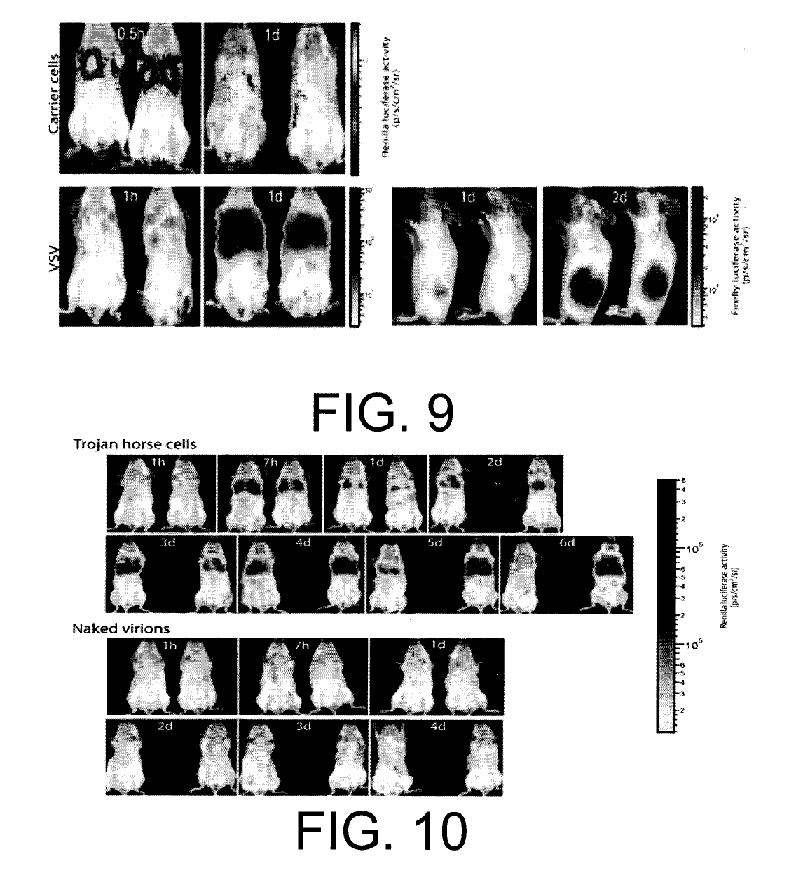

FIG. 9 illustrates, interestingly, that some of the A549^{RLUC} cells were able to bypass the lung capillary bed and delivery virus to subcutaneous tumours. Dual-enzyme bioluminescent imaging illustrating VSV delivery to established CT26 lung (left panels) or subcutaneous (right panel) tumors using the human A549 lung carcinoma cell line as described above with respect to FIG. 8 for L1210 cells.

This Example demonstrate that it is possible to use cellular carrier vehicles from different species and tissues of origin to deliver virus to wide spread tumour sites in mice and that cell lineage, tissue of origin or other characteristics of the carrier cell can be adapted or selected to determine the biodistribution of the delivery vehicle.

### Example 4

### Trojan Horse Delivery of Virus to Tumors Beds in the Presence of Circulating Antibody

The ability of infected Trojan Horse carrier cells to deliver virus to tumours in the face of sterilizing immunity was tested.

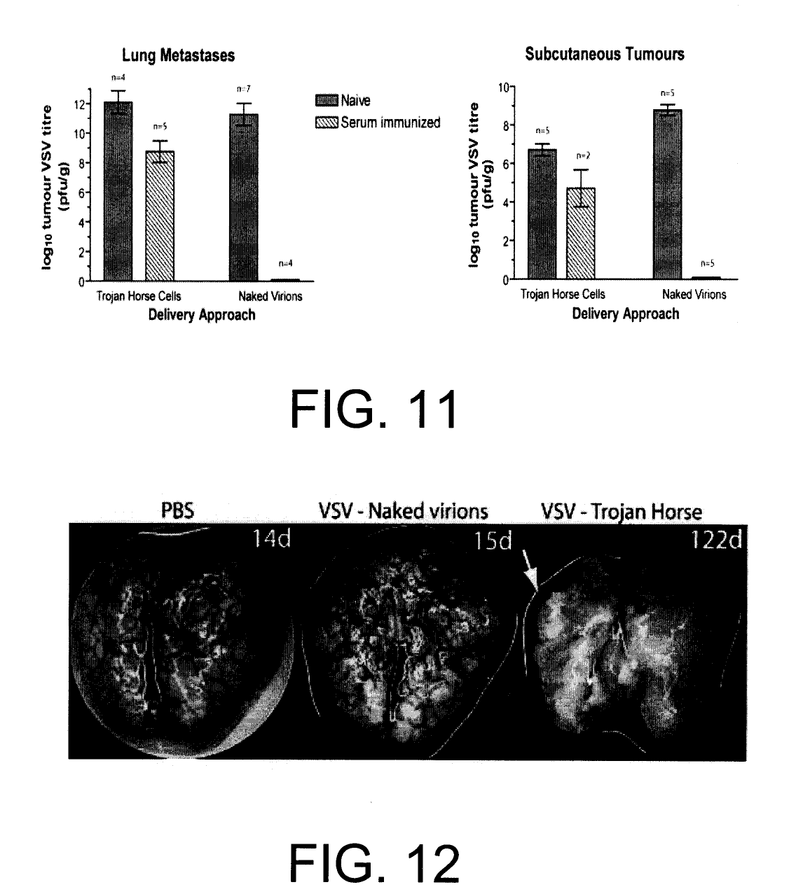

FIG. 10, FIG. 11 and FIG. 12 show that when virus alone (herein refereed to as “naked virus”) is infused into animals with circulating antibodies, the virus is unable to target tumours in the lung. The majority of the virus encoded luciferase signal is found transiently in the spleen of naked virus treated animals and is eliminated within 24 hours of infection. This likely reflects clearance of virus by immune cells armed with antibodies. In contrast VSV^{FLUC} delivered by infusion of infected Trojan Horse carrier cells is able to infect lung tumours in immune mice where it persists and continues to replicate for up to 6 days. The absolute amount of virus replicating in lung and subcutaneous tumours was evaluated in naïve and immune animals by homogenization of tumours and direct plaque assay.

FIG. 10 illustrates an imaging of tumor infection in lung tumor-bearing hosts with pre-existing antibodies against VSV. Following passive immunization, mice were treated with a single dose of 10⁶ CT26 tumor cells infected with VSV^{FLUC} (upper panels) or with 5×10⁸ pfu naked VSV^{FLUC} virions (lower panels). Virus delivery and tumor infection were followed via bioluminescent imaging. Luciferase activity is comparison of animals infused with carrier cells, and the color bar indicates Renilla activity (p/s/cm²/sr).

FIG. 11 illustrates quantitative analysis comparing VSV titers in CT26 tumors at 24 h following systemic treatment of mice with (shaded bars) or without (hatched bars) pre-existing VSV antibodies. Lung (left) or subcutaneous (right) tumor-bearing mice were treated with either 10⁶ in vitro-infected CT26 cells or 5×10⁸ naked virions. Mean log₁₀ tumor titers+/−SD are plotted.

These data indicate that naked virus is unable to be delivered and replicate in tumours in the presence of neutralizing antibodies, which is consistent with imaging studies. In contrast, virus can be delivered by infected carrier cells in immune, tumour bearing mice and though it is less efficient than in naïve mice, it is still some 6-8 logs greater than in mice treated with naked virus. Immune animals that were treated with naked virus did not thrive and were euthanized 14-15 days following the initiation of viral therapy due to respiratory distress.

FIG. 12 illustrates lung tumor burden in VSV-immune mice, following treatment with either naked VSV particles or Trojan Horse carrier cells. At 6 days following lung tumor seeding in balb/c mice with pre-existing immunity to VSV, intravenous treatment was initiated with either PBS, 5×10⁸ pfu VSV or 10⁶ VSV-infected CT26 Trojan Horse carrier cells. Mice were dosed 3 times per week for 4 weeks or until reaching defined endpoint criteria (respiratory distress, weight loss, etc.) Representative photographs show lung tumor burden in PBS- and VSV-treated mice when sacrificed at endpoint (14 and 15 days post-treatment start, respectively). Trojan Horse carrier treated animal remained disease-free until sacrifice at 122 days at which point lungs were imaged. arrow indicates a residual tumor nodule.

These data illustrate the lungs of these animals at autopsy. Extensive disease with hundreds of visible nodules is shown for the control (PBS), and VSV treatment with naked virions. In contrast, lungs removed 120 days following initiation of Trojan Horse therapy in an immune animal had very limited disease, and as depicted in FIG. 12, only one visible tumour nodule is found.

### Example 5

### Delivery of Invertebrate Carrier Cells to Mice

Insect cells may be used for delivery of virus to mice. In this example, insect cells were infected with VSV luciferase, and subsequently injected into mice and imaged. SF-9 is an insect cell line derived from the pupal ovary of Spondoptera frugiperda. This Example shows that a xenogenic insect carrier cell line can deliver an oncolytic virus to a target by systemic administration.

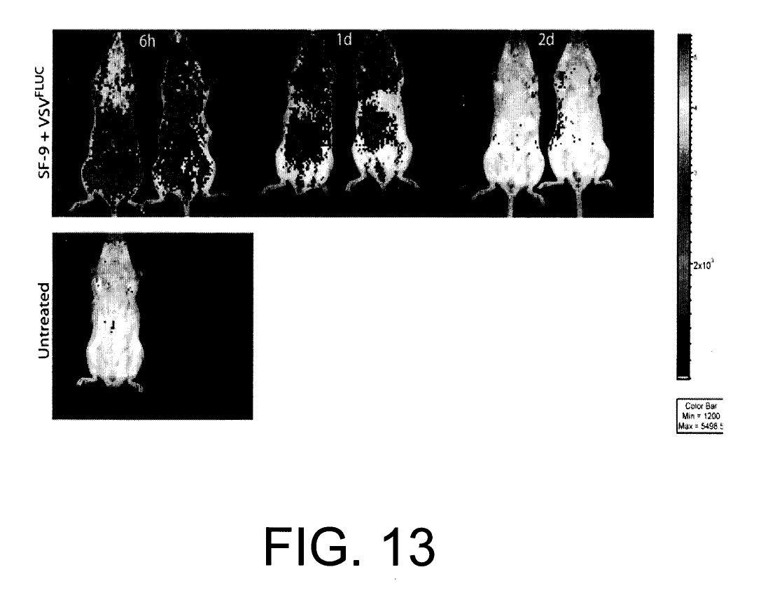

FIG. 13 illustrates the dissemination of invertebrate carrier cells in mice following intravenous administration. Following 3 hours of in vitro infection with VSV^{FLUC}, 10⁶ SF-9 insect cells were injected into the tail vein of each mouse. Bioluminescence imaging was performed at the indicated timepoints of 6 h, 1 d, and 2 d post-injection to assess the distribution of virus carrying cells.

The mice depicted are duplicate mice, neither of which has any tumors. This example shows the distribution of infected cells following IV administration, indicating the feasibility of administering such cells for circulation and delivery to established tumors.

As evidenced by a widely disseminated presence of luciferase after 6 h and 1 d, the insect cells do not lodge in the lungs or elsewhere, but are disseminated evenly through the animal, and cleared by day 2. Thus, tumors anywhere in the body can be targeted using this model. Further, not only does the small size of the cells contribute to the wide distribution, but because these xenogenic insect cells do not lodge in any particular tissue due to surface recognition, they act essentially as “blank slates” that could potentially be programmed or modified to attach to particular sites in the body. In alternative embodiments, a carrier cell such as a SF-9 carrier cell, can be modified, for example by transformation, to target the cells to specific tissues in the body, such as tumour cells.

In selected embodiments, carrier cells may be selected so that infected carrier cells are adapted to pass through microcapillary beds, such as microcapillary beds in the lungs.

### Example 6

### Susceptibility of Human Sarcoma Cell Lines to Vesicular Stomatitis Virus Infection Post Trichostatin A Treatment

Sarcomas are a heterogeneous group of malignant tumors with different histopathologies, sites of presentation and age distribution. They originate from mesenchymal tissues and tend to occur in a sporadic fashion (Wang (2005). The Cancer Journal, July-August; 11(4):294-3056). The mainstay of treatment for local disease control is wide margin surgical resection. Despite advancements in local control management, deep high grade sarcomas have up to a 50% metastatic relapse rates.

In vitro carcinoma models have proven to be susceptible targets for oncolytic VSV infection, which may be due to the presence of an impaired innate interferon pathway in carcinoma cells (Barber (2005). Oncogene 24, 7710-7719). Similarly, in vitro studies using sarcoma biopsy specimens show that they are more susceptible to VSV infection than normal tissue specimens.

Histone deacetylase inhibitors (HDACI), can block interferon production at the transcriptional level (Chang et al. (2004). Proc Natl Acad Sci USA Jun. 29, 9578-83). Examples of these compounds are Trichostatin A (TSA) and Valproic acid. This Example illustrates that treating sarcoma cells with TSA can enhance their susceptibility to VSV infection at a relatively low viral concentration, without significantly affecting the susceptibility of normal cells that are also treated with TSA. Accordingly, in some embodiments the invention involves concomitant treatment with an oncolytic virus and an agent, such as an HDACI, that increases the susceptibility of a target cell, such as a sarcoma or carcinoma cell, to an oncolytic virus, such as VSV.

The following steps have been used for Osteosarcoma (U2OS), Ewing's Sarcoma (19304) and skeletal muscle cell lines:
1. Cells have been cultured from human biopsy specimens and preserved in cryotubes containing 10% Dimethyl Sulfoxide (DMSO).
2. Cryotubes are kept in a −80° C. environment.
3. To carry out our experiments, samples in the cryotubes are thawed and transferred to a 15 cm culture dish.
4. Dulbecco's Modified Eagle's Medium (DMEM) was added to the culture dish which was then incubated in a 37° C. environment.
5. Light microscopy was used to confirm that >90% of the culture plate was confluent before proceeding to the next step.
6. Cells were then collected from the culture dish, using 0.05% porcine trypsine, and transferred in equal amount into 4 well plates.
7. Cells were incubated again in a 37° C. environment using DMEM.
8. Light microscopy was used to confirm that each well of the culture plate was >90% confluent before proceeding to the next step.
9. Trichostatin A was then added to the wells in the following pattern.
10. After 6 hours, the media from each well was removed and VSV, encoding the gene for GFP, was then added to the wells in the following pattern.
11. VSV is left to infect the cells for 48 hrs while incubated in 370 C. environment.
12. Wells were examined under a fluorescent light microscopy to evaluate GFP production.

Pictures were obtained from the fluorescent light microscope using consistent magnification and exposure time.

Osteosarcoma cell line (U2OS) photographs were taken 48 hours post infection. Compared to the control, moderate GFP production is evident after infecting the U2OS cells with VSV MOI 0.01. However, there is considerably more GFP production in the infected cells that have been pre-treated with TSA either at TSA 100 ng/ml+VSV MOI 0.01 or TSA 300 ng/ml+VSV MOI 0.01.

Ewing's Sarcoma cell line photographs were taken 48 hours post infection. Compared to the control, moderate GFP production is evident after infecting the 19304 cells with VSV MOI 0.001. However, there is considerably more GFP production in the infected cells that have been pre-treated with TSA either at TSA 100 ng/ml+VSV MOI 0.001 or at TSA 300 ng/ml+VSV MOI 0.001.

Normal muscle cell line photographs were taken 72 hours post infection. Compared to the control, there is minimal GFP production evident after infecting the TSA treated and untreated muscle cells with VSV MOI 0.01. Observations were made for TSA 100 ng/ml+VSV MOI 0.01 and for TSA 300 ng/ml+VSV MOI 0.01.

This data collected in connection with this Example illustrates the use of an adjunct treatment that can enhance the infective potential of VSV towards sarcoma cells, such as osteosarcoma and Ewing's sarcoma.

### Example 7

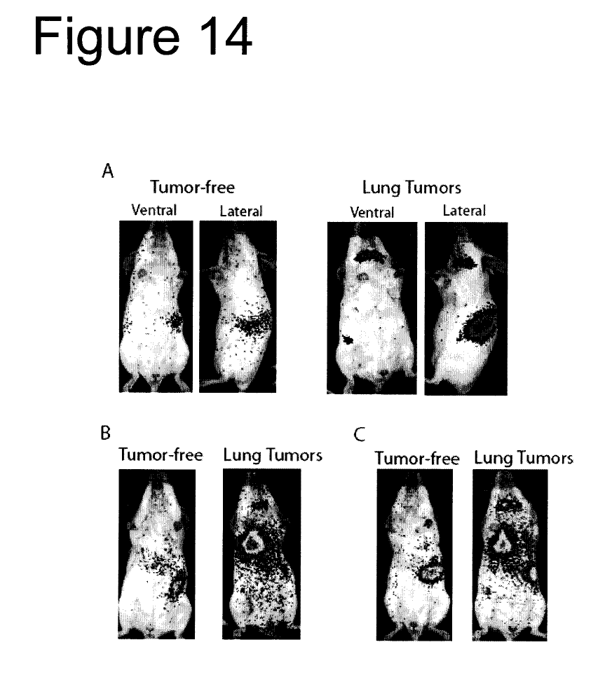

FIG. 14 illustrates the biodistribution of systemically administered human leukemia carrier cells. Human leukemia carrier cell lines were infected with VSV-FLuc at an MOI of 10 for 2 h. Tumor-free or CT26 lung tumor-bearing mice were then intravenously treated with 10⁶ infected cells. The localization of carrier cells was determined 2.5 h following treatment via in vivo molecular imaging to detect fluc-generated bioluminescence. Localization of Jurkat T-lymphocytic leukemia (A), K562 myeloid leukemia (B), and Meg-01 myeloid leukemia (C) carrier cells are shown.

Whereas lymphocytic leukemia cells were found to accumulate exclusively within the spleen and lymph nodes of both tumor-free and lung tumor-bearing mice (A), myeloid carrier cell lines have a specific affinity for tumor-bearing lung tissue (B,C). Extensive accumulation of K562 and Meg-01 cells expressing virus-encoded luciferase was seen in the lungs of mice when tumors were present (B,C, right images), but not in tumor free mice (fig B,C, left images).

### REFERENCES

Ahmed, M, Cramer, S D and Lyles, D S (2004). Sensitivity of prostate tumors to wild type and M protein mutant vesicular stomatitis viruses. Virology 330: 34-49.
Bell et al. (2003) Getting oncolytic virus therapies off the ground. Cancer. Cell. July; 4(1):7-11.
Bischoff, J R et al. (1996). An adenovirus mutant that replicates selectively in p53-deficient human tumor cells. Science 274: 373-376.
Campbell, R E et al. (2002). A monomeric red fluorescent protein. Proc Natl Acad Sci USA 99: 7877-7882.
Carlos, T M and Harlan, J M (1994). Leukocyte-endothelial adhesion molecules. Blood 84: 2068-2101.
Chen, Y, Yu, D C, Charlton, D and Henderson, D R (2000). Pre-existent adenovirus antibody inhibits systemic toxicity and antitumor activity of CN706 in the nude
Chillon, M, Lee, J H, Fasbender, A and Welsh, M J (1998). Adenovirus complexed with polyethylene glycol and cationic lipid is shielded from neutralizing antibodies in vitro. Gene Therapy 5: 995-1002.
Chiocca, E. A. et al. (2004). A phase I open-label, dose-escalation, multi-institutional trial of injection with an E1B-attenuated adenovirus, ONYX-015, into the peritumoral region of recurrent malignant gliomas, in the adjuvant setting. Mol. Ther. 10, 958-966.
Coffey, M C, Strong, J E, Forsyth, P A and Lee, P W (1998). Reovirus therapy of tumors with activated Ras pathway. Science 282: 1332-1334.
Cole, C et al. (2005). Tumor-targeted, systemic delivery of therapeutic viral vectors using hitchhiking on antigen-specific T cells. Nat Med 11: 1073-1081.
Coukos, G et al. (1999). Use of carrier cells to deliver a replication-selective herpes simplex virus-1 mutant for the intraperitoneal therapy of epithelial ovarian cancer. Clin Cancer Res 5: 1523-1537.
Crittenden, M et al. (2003). Pharmacologically regulated production of targeted retrovirus from T cells for systemic antitumor gene therapy. Cancer Res 63: 3173-3180.
Ebert, O, Harbaran, S, Shinozaki, K and Woo, S L (2005). Systemic therapy of experimental breast cancer metastases by mutant vesicular stomatitis virus in immune-competent mice. Cancer Gene Ther 12: 350-358.
Everts and van der Poel H G (2005). Replication-selective oncolytic viruses in the treatment of cancer. Cancer Gene Ther. February; 12(2):141-161.
Fisher, K D et al. (2001). Polymer-coated adenovirus permits efficient retargeting and evades neutralising antibodies. Gene Therapy 8: 341-348.
Garcia-Castro, J et al. (2005). Tumor cells as cellular vehicles to deliver gene therapies to metastatic tumors. Cancer Gene Ther 12: 341-349.
Gobet, R, Cerny, A, Ruedi, E, Hengartner, H and Zinkernagel, R M (1988). The role of antibodies in natural and acquired resistance of mice to vesicular stomatitis virus. Exp Cell Biol 56: 175-180.
Green, N K et al. (2004). Extended plasma circulation time and decreased toxicity of polymer-coated adenovirus. Gene Therapy 11: 1256-1263.
Grote, D et al. (2001). Live attenuated measles virus induces regression of human lymphoma xenografts in immunodeficient mice. Blood 97: 3746-3754.
Harrington, K et al. (2002). Cells as vehicles for cancer gene therapy: the missing link between targeted vectors and systemic delivery? Hum Gene Ther 13: 1263-1280.
Harrow, S. et al. (2004). HSV1716 injection into the brain adjacent to tumour following surgical resection of high-grade glioma: safety data and long-term survival. Gene Ther. 11, 1648-1658.
Hirasawa, K et al. (2003). Systemic reovirus therapy of metastatic cancer in immune-competent mice. Cancer Res 63: 348-353.
Hirasawa, K. et al. (2003). Systemic reovirus therapy of metastatic cancer in immune-competent mice. Cancer Res. 63, 348-353.
Hummel, J L, Safroneeva, E and Mossman, K L (2005). The role of ICPO-null HSV-1 and interferon signaling defects in the effective treatment of breast adenocarcinoma. Mol Ther 12: 1101-1110.
Ikeda, K. et al. (1999) Oncolytic virus therapy of multiple tumors in the brain requires suppression of innate and elicited antiviral responses. Nature Med. 5, 881-887.
Jevremovic, D et al. (2004). Use of blood outgrowth endothelial cells as virus-producing vectors for gene delivery to tumors. Am J Physiol Heart Circ Physiol 287: H494H500.
Kaufman, H. L. et al. (2005). Targeting the local tumor microenvironment with vaccinia virus expressing B7.1 for the treatment of melanoma. J. Clin. Invest. 115, 1903-1912.
Komarova, S, Kawakami, Y, Stoff-Khalili, M A, Curiel, D T and Pereboeva, L (2006). Mesenchymal progenitor cells as cellular vehicles for delivery of oncolytic adenoviruses. Mol Cancer Ther 5: 755-766.
Lang, S I, Giese, N A, Rommelaere, J, Dinsart, C and Cornelis, J J (2006). Humoral immune responses against minute virus of mice vectors. J Gene Med 12: 12-1222.
Lorence, R. M. et al. (2003). Overview of phase I studies of intravenous administration of PV701, an oncolytic virus. Curr. Opin. Mol. Ther. 5, 618-624.
Lorence, R. M. et al. (2005). Continuing the interaction between non-clinical and clinical studies. Third International Meeting on Oncolytic Virus Therapeutics: Banff, Alberta (12 Mar. 2005).
Mastrangelo, M. J. et al. (1999) Intratumoral recombinant GM-CSF-encoding virus as gene therapy in patients with cutaneous melanoma. Cancer Gene Ther. 6(5), 409-422.
Matthews, C, Jenkins, G, Hilfinger, J and Davidson, B (1999). Poly-L-lysine improves gene transfer with adenovirus formulated in PLGA microspheres. Gene Therapy 6: 1558-1564.
McCart et al. (2001). Systemic Cancer Therapy with a Tumor-selective Vaccinia Virus Mutant Lacking Thymidine Kinase and Vaccinia Growth Factor Genes. Cancer Research 61, 8751-8757).
Mineta, T, Rabkin, S D, Yazaki, T, Hunter, W D and Martuza, R L (1995). Attenuated multi-mutated herpes simplex virus-1 for the treatment of malignant gliomas. Nat Med 1: 938-943.
mouse LNCaP xenograft model: implications and proposals for human therapy. Hum Gene Ther 11: 1553-1567.
Myers, R. et al. (2005). Oncolytic activities of approved mumps and measles vaccines for therapy of ovarian cancer. Cancer Gene Ther. 12, 593-599.
Naldini, L et al. (1996). In vivo gene delivery and stable transduction of nondividing cells by a lentiviral vector. Science 272: 263-267.
O'Riordan, C R et al. (1999). PEGylation of adenovirus with retention of infectivity and protection from neutralizing antibody in vitro and in vivo. Hum Gene Ther 10: 1349-1358.
Obuchi, M, Fernandez, M and Barber, G N (2003). Development of recombinant vesicular stomatitis viruses that exploit defects in host defense to augment specific oncolytic activity. J Virol 77: 8843-8856.
Parato et al. (2005). Recent progress in the battle between oncolytic viruses and tumours. Nat Rev Cancer. 5(12): 965-76.
Parato, K A, Senger, D, Forsyth, P A and Bell, J C (2005). Recent progress in the battle between oncolytic viruses and tumours. Nat Rev Cancer 5: 965-976.
Pearce, O M, Fisher, K D, Humphries, J, Seymour, L W, Smith, A and Davis, B G (2005). Glycoviruses: chemical glycosylation retargets adenoviral gene transfer. Angew Chem Int Ed Engl 44: 1057-1061.
Pecora, A. L. et al. (2002) Phase I trial of intravenous administration of PV701, an oncolytic virus, in patients with advanced solid cancers. J. Clin. Oncol. 20, 2251-2266.
Plaksin, D, Porgador, A, Vadai, E, Feldman, M, Schirrmacher, V and Eisenbach, L (1994). Effective anti-metastatic melanoma vaccination with tumor cells transfected with MHC genes and/or infected with Newcastle disease virus (NDV). Int J Cancer 59: 796-801.
Raykov, Z, Balboni, G, Aprahamian, M and Rommelaere, J (2004). Carrier cell-mediated delivery of oncolytic parvoviruses for targeting metastases. Int J Cancer 109: 742-749.
Reid et al. (2002). Intravascular adenoviral agents in cancer patients: lessons from clinical trials. Cancer Gene Ther. 9, 979-986.
Reid, T. et al. (2001). Intra-arterial administration of a replication selective adenovirus (d11520) in patients with colorectal carcinoma metastatic to the liver: a phase I trial. Gene Ther. 8, 1618-1626.
Ries S. J., Brandts C. H. (2004) Oncolytic viruses for the treatment of cancer: current strategies and clinical trials. Drug Discov. Today 2004 Sep. 1; 9(17):759-768.
Schiedner, G et al. (2003). A hemodynamic response to intravenous adenovirus vector particles is caused by systemic Kupffer cell-mediated activation of endothelial cells. Hum Gene Ther 14: 1631-1641.
Shah et al., (2003). Oncolytic viruses: clinical applications as vectors for the treatment of malignant gliomas. J. Neurooncol. 65, 203-226.
Skalak, R and Branemark, P I (1969). Deformation of red blood cells in capillaries. Science 164: 717-719.
Stein, J V and Nombela-Arrieta, C (2005). Chemokine control of lymphocyte trafficking: a general overview. Immunology 116: 1-12.
Stojdl, D. F. et al. (2000). Exploiting tumor-specific defects in the interferon pathway with a previously unknown oncolytic virus. Nature Med. 6, 821-825.
Stojdl, D. F. et al. (2003) VSV strains with defects in their ability to shutdown innate immunity are potent systemic anti-cancer agents. Cancer Cell 4, 263-275.
Thorne, S H, Negrin, R S and Contag, C H (2006). Synergistic antitumor effects of immune cell-viral biotherapy. Science 311: 1780-1784.
Tsai, V et al. (2004). Impact of human neutralizing antibodies on antitumor efficacy of an oncolytic adenovirus in a murine model. Clin Cancer Res 10: 7199-7206.
Wakimoto et al. (2004) Altered expression of antiviral cytokine mRNAs associated with cyclophosphamide's enhancement of viral oncolysis. Gene Ther. 11, 214-23.
Wakimoto, H et al. (2002). The complement response against an oncolytic virus is species-specific in its activation pathways. Mol Ther 5: 275-282.
Worgall, S, Wolff, G, Falck-Pedersen, E and Crystal, R G (1997). Innate immune mechanisms dominate elimination of adenoviral vectors following in vivo administration. Hum Gene Ther 8: 37-44.
Xia, Z. J. et al. (2004). Phase III randomized clinical trial of intratumoral injection of E1B gene-deleted adenovirus (H101) combined with cisplatin-based chemotherapy in treating squamous cell cancer of head and neck or esophagus. Ai Zheng 23, 1666-1670.
Ye, X, Jerebtsova, M and Ray, P E (2000). Liver bypass significantly increases the transduction efficiency of recombinant adenoviral vectors in the lung, intestine, and kidney. Hum Gene Ther 11: 621-627.

1. A method for treating a neoplastic disease in a mammalian subject, comprising:
a) providing a carrier cell that is capable of localizing within a therapeutic targeting interval to a diseased tissue in the subject, the diseased tissue comprising a target cell that is characteristic of the neoplastic disease, the carrier cell being allogenic or xenogenic with respect to the target cell;
b) infecting the carrier cell with an oncolytic virus to produce an infected carrier cell, wherein the oncolytic virus is capable of productive lytic replication in the infected carrier cell following the therapeutic targeting interval;
c) administering the infected carrier cell to the subject when the infection of the carrier cell by the oncolytic virus is in an eclipse phase, and so that the subject does not mount an effective adaptive immune response to the infected carrier cell within the therapeutic targeting interval; and,
d) permitting lysis of the infected carrier cell by the oncolytic virus in the diseased tissue following the conclusion of the therapeutic targeting interval, to produce an infective secondary oncolytic virus that infects and kills the target cell by productive lytic replication, and so that the lysis of the infected carrier cell is followed by an adaptive immune response in the subject against antigenic determinants on the carrier cell.

2. The method of claim 1, wherein the subject exhibits an adaptive immune response against antigenic determinants on the oncolytic virus prior to administering the infected carrier cell.

3. The method of claim 1, wherein the carrier cell has an affinity for a neoplastic cell or tissue in the subject.

4. The method of claim 1, wherein the administration of the infected carrier cell is by intratumoral injection.

5. The method of claim 1, wherein the administration of the infected carrier cell is by a systemic route of administration.

6. The method of claim 5, wherein the systemic route of administration is a parenteral administration.

7. The method of claim 6, wherein the parenteral administration is an intravenous injection.

8. The method of claim 1, wherein the neoplastic disease is a cancer.

9. The method of claim 1, wherein the diseased tissue is a solid tumor.

10. The method of claim 1, wherein the carrier cell is an insect cell, a reptile cell, an amphibian cell, an avian cell or a mammalian cell.

11. The method of claim 1, wherein the carrier cell is an SF9 cell, a CT26 cell, an A549 cell, a K562 cell, a Meg01 cell, a Jurkat cell, or a L1210 cell.

12. The method of claim 1, wherein the carrier cell is an immortalized cell.

13. The method of claim 1, wherein the carrier cell is a neoplastic cell derived from a tumor.

14. The method of claim 1, wherein the oncolytic virus is a DNA virus, a positivesense RNA virus, a negativesense RNA virus, or a double stranded RNA virus.

15. The method of claim 1, wherein the infected carrier cell is adapted so as to pass through a pulmonary microcapillary bed in the subject.

16. The method of claim 1, wherein the subject is a human.

17. The method of claim 1, further comprising a subsequent step of treatment, comprising:
a) providing a second carrier cell that is capable of localizing within a second therapeutic targeting interval to the diseased tissue in the subject, the diseased tissue comprising a second target cell that is characteristic of the neoplastic disease, the second carrier cell being allogenic or xenogenic with respect to the target cell, the second target cell, and with respect to the carrier cell;
b) infecting the second carrier cell with a second oncolytic virus, the second oncolytic virus being the same or different than the oncolytic virus, to produce a second infected carrier cell, wherein the second oncolytic virus is capable of productive lytic replication in the second infected carrier cell following the second therapeutic targeting interval;
c) administering the second infected carrier cell to the subject when the infection of the second carrier cell by the second oncolytic virus is in an eclipse phase, and so that the subject does not mount an effective adaptive immune response to the second infected carrier cell within the second therapeutic targeting interval; and,
d) permitting lysis of the second infected carrier cell by the second oncolytic virus in the diseased tissue at the conclusion of the second therapeutic targeting interval, to produce an infective tertiary oncolytic virus that infects and kills the second target cell byproductive lytic replication, and so that the lysis of the second infected carrier cell is followed by an adaptive immune response in the subject against antigenic determinants on the second carrier cell.

18. The method of claim 17, further comprising additional subsequent treatment steps with alternative antigenically distinct carriers.

19. A method for treating a neoplastic disease in a mammalian subject comprising administering a carrier cell to the subject, wherein:
a) the carrier cell is capable of localizing within a therapeutic targeting interval to a diseased tissue in the subject, the diseased tissue comprising a target cell that is characteristic of the neoplastic disease, the carrier cell being allogenic or xenogenic with respect to the target cell;
b) the carrier cell is infected in vitro with an oncolytic virus to produce an infected carrier cell, wherein the oncolytic virus is capable of productive lytic replication in the infected carrier cell following the therapeutic targeting interval;
c) the infection of the carrier cell by the oncolytic virus is in an eclipse phase for administration to the subject, so that the subject does not mount an effective adaptive immune response to the infected carrier cell within the therapeutic targeting interval; and,
d) lysis of the infected carrier cell by the oncolytic virus in the diseased tissue occurs at the conclusion of the therapeutic targeting interval, to produce an infective secondary oncolytic virus that infects and kills the target cell by productive lytic replication, and so that the lysis of the infected carrier cell is followed by an adaptive immune response by the subject against antigenic determinants on the carrier cell.

20. An infected carrier cell, produced by infecting a carrier cell with an oncolytic virus, for treating a neoplastic disease in a mammalian subject, wherein:
a) the carrier cell is capable of localizing within a therapeutic targeting interval to a diseased tissue in the subject, the diseased tissue comprising a target cell that is characteristic of the neoplastic disease, the carrier cell being allogenic or xenogenic with respect to the target cell;
b) the oncolytic virus is capable of productive lytic replication in the infected carrier cell following the therapeutic targeting interval;
c) the infection of the carrier cell by the oncolytic virus is in an eclipse phase, so that the subject does not mount an effective adaptive immune response to the infected carrier cell within the therapeutic targeting interval; and,
d) lysis of the infected carrier cell by the oncolytic virus in the diseased tissue occurs at the conclusion of the therapeutic targeting interval, to produce an infective secondary oncolytic virus that infects and kills the target cell by productive lytic replication, and so that the lysis of the infected carrier cell is followed by an adaptive immune response by the subject against antigenic determinants on the carrier cell.

21. The infected carrier cell of claim 20, wherein the carrier cell is an insect cell, a reptile cell, an amphibian cell, an avian cell or a mammalian cell.

22. The infected carrier cell of claim 20, wherein the carrier cell is an SF9 cell, a CT26 cell, an A549 cell, a K562 cell, a Meg01 cell, a Jurkat cell, or a L1210 cell.

23. The infected carrier cell of claim 20, wherein the carrier cell is an immortalized cell.

24. The infected carrier cell of claim 20, wherein the carrier cell is a neoplastic cell derived from a tumor.

25. The infected carrier cell of claim 20, wherein the oncolytic virus is a DNA virus, a positivesense RNA virus, a negativesense RNA virus, or a double stranded RNA virus.

26. The infected carrier cell of claim 20, wherein the infected carrier cell is adapted so as to pass through a pulmonary microcapillary bed in the subject.

27. A method of formulating a medicament for treating a neoplastic disease in a mammalian subject, comprising:
a) selecting a carrier cell that is capable of localizing within a therapeutic targeting interval to a diseased tissue in the subject, the diseased tissue comprising a target cell that is characteristic of the neoplastic disease, the carrier cell being allogenic or xenogenic with respect to the target cell;
b) infecting the carrier cell in vitro with an oncolytic virus to produce an infected carrier cell, wherein the oncolytic virus is capable of productive lytic replication in the infected carrier cell following the therapeutic targeting interval;
c) formulating the medicament for administration to the subject when the infection of the carrier cell by the oncolytic virus is in an eclipse phase, so that the subject will not mount an effective adaptive immune response to the infected carrier cell within the therapeutic targeting interval; and,
d) adapting the formulation so that lysis of the infected carrier cell by the oncolytic virus in the diseased tissue occurs at the conclusion of the therapeutic targeting interval, to produce an infective secondary oncolytic virus that infects and kills the target cell by productive lytic replication, and so that the lysis of the infected carrier cell will be followed by an adaptive immune response by the subject against antigenic determinants on the carrier cell.
